(19) United States
(12) Patent Application Publication
(10) Pub. No.: US 2002/0164314 A1
(43) Pub. Date: Nov. 7, 2002

(54) Ovarian hormone induced neural stem cell increase

(75) Inventors: Samuel Weiss, Calgary (CA); Tetsuro Shingo, Okayama (JP)

Correspondence Address: BURNS DOANE SWECKER & MATHIS L L P, POST OFFICE BOX 1404, ALEXANDRIA, VA, 22313-1404, US

(73) Assignee: Neurostasis, Inc., CALGARY, AB (CA)

(21) Appl. No.: 10/084,675

(22) Filed: Feb. 28, 2002

Related U.S. Application Data
(60) Provisional application No. 60/272,940, filed on Mar. 2, 2001.

(51) Int. Cl. A61K 48/00; C12N 5/08; A61K 31/56

(52) U.S. Cl. 424/93.21; 424/93.7; 435/368; 514/182

(57) ABSTRACT

This invention provides a method of increasing the number of neural stem cells by using ovarian hormones. Ovarian hormones induce an increase in the number of neural stem cells, resulting in a larger pool of neural stem cells, which may be used in the treatment or amelioration of neurodegenerative diseases or conditions. Another aspect of the invention provides a method for identifying genes that regulate the ovarian hormone-induced stem cell increase.

### RELATED APPLICATION

This application claims the benefit of U.S. Provisional Application Serial No. 60/272,940, filed Mar. 2, 2001, which is hereby incorporated by reference in its entirety.

### FIELD OF THE INVENTION

This invention relates to a method of increasing multipotent neural stem cells by using ovarian hormones, a method for treating or ameliorating neurodegenerative diseases or conditions, as well as a method for identifying genes which are induced by ovarian hormones in stem cell.

### REFERENCES

U.S. Pat. No. 5,750,376.

U.S. Pat. No. 5,843,934.

U.S. Pat. No. 5,851,832.

U.S. Pat. No. 5,980,885.

WO 99/21996.

Alonso, G., “Prolonged corticosterone treatment of adult rats inhibits the proliferation of oligodendrocyte progenitors present throughout white and gray matter regions of the brain”, GLIA 31: 219-231 (2000).

Baniahmad et al., “Enhancement of human estrogen receptor activity by SPT6: a potential coactivator”, Mol. Endocrinol. 9(1):34-43 (1995).

Hidalgo A. et al., “Estrogen and non-estrogenic ovarian influences combine to promote the recruitment and decrease the turnover of new neurons in the adult female canary brain”, J. Neurobiol. 27(4): 470-487 (1995).

Tanapat, P. et al., “Estrogen stimulates a transient increase in the number of new neurons in the dentate gyrus of the adult female rat”, J. Neuroscience 19(14): 5792-5801 (1999).

All of the above publications, patents and patent applications are herein incorporated by reference in their entirety to the same extent as if the disclosure of each individual publication, patent application or patent was specifically and individually indicated to be incorporated by reference in its entirety.

### BACKGROUND OF THE INVENTION

In recent years, neurodegenerative disease has become an important concern due to the expanding elderly population which is at greatest risk for these disorders.

Neurodegenerative diseases include the diseases which have been linked to the degeneration of neural cells in particular locations of the central nervous system (CNS), leading to the inability of these cells to carry out their intended function. These diseases include Alzheimer's Disease, Multiple Sclerosis (MS), Huntington's Disease, Amyotrophic Lateral Sclerosis, and Parkinson's Disease. In addition, probably the largest area of CNS dysfunction (with respect to the number of affected people) is not characterized by a loss of neural cells but rather by abnormal functioning of existing neural cells. This may be due to inappropriate firing of neurons, or the abnormal synthesis, release, and processing of neurotransmitters. These dysfunctions may be the result of well studied and characterized disorders such as depression and epilepsy, or less understood disorders such as neurosis and psychosis. Moreover, brain injuries often result in the loss of neural cells, the inappropriate functioning of the affected brain region, and subsequent behavior abnormalities.

Consequently, it is desirable to supply neural cells to the brain to compensate for degenerate or lost neurons in order to treat neurodegenerative diseases or conditions. One approach to this end is to transplant neural cells into the brain of the patient. This approach requires a source of large amounts of neural cells, preferably from the same individual or a closely related individual such that host-versus-graft or graft-versus-host rejections can be minimized. As it is not practical to remove a large amount of neurons or glial cells from one person to transplant to another, a method to culture large quantity of neural cells is necessary for the success of this approach.

Another approach is to induce the production of neural cells in situ to compensate for the lost or degenerate cells. This approach requires extensive knowledge about whether it is possible to produce neural cells in brains, particularly adult brains, and how.

The development of techniques for the isolation and in vitro culture of multipotent neural stem cells (for example, see U.S. Pat. Nos. 5,750,376; 5,980,885; 5,851,832) significantly increased the outlook for both approaches. It was discovered that fetal brains can be used to isolate and culture multipotent neural stem cells in vitro. Moreover, in contrast to the long time belief that adult brain cells are not capable of replicating or regenerating brain cells, it was found that neural stem cells may also be isolated from brains of adult mammals. These stem cells, either from fetal or adult brains, are capable of self-replicating. The progeny cells can again proliferate or differentiate into any cell in the neural cell lineage, including neurons, astrocytes and oligodendrocytes. Therefore, these findings not only provide a source of neural cells which can be used in transplantations, but also demonstrate the presence of multipotent neural stem cells in adult brain and the possibility of producing neurons or glial cells from these stem cells in situ.

It is therefore desirable to develop methods of efficiently increasing the number of neural stem cells for two purposes: to obtain more stem cells and hence neural cells which can be used in transplantation therapies, and to identify methods which can be used to produce more stem cells in situ.

### SUMMARY OF THE INVENTION

This invention provides a method of increasing the number of neural stem cells by using ovarian hormones. It was found unexpectedly that pregnant mice had more neural stem cells than their virgin counterparts. The role of ovarian hormones was further confirmed by ovarectomy experiments, which indicate that removal of the ovaries resulted in reduced number of neural stem cells. Therefore, ovarian hormones can be used to increase the number of neural stem cells. Another aspect of the invention provides a method for identifying genes that are induced or suppressed by ovarian hormones in neural stem cells.

Accordingly, an aspect of the present invention provides a method of increasing neural stem cells, comprising providing an ovarian hormone to at least one neural stem cell under conditions which result in an increase in the number of neural stem cells. The neural stem cell is preferably located in the brain of an animal. More preferably, the neural stem cell is located in the subventricular zone of the brain. Most preferably, the animal is an adult animal. The ovarian hormone can be provided in the proximity of the neural stem cell, and is preferably administered to a ventricle, particularly a lateral ventricle, of the brain. It is also contemplated that the method can be applied to neural stem cells in an in vitro culture to which the ovarian hormone is provided.

The ovarian hormone is preferably an estrogen, a progestin, or a combination of both. The ovarian hormone may also be a combination of different estrogens or a combination of different progestins.

Another aspect of the present invention provides a method of identifying a gene which participates in neural stem cell increase, comprising:

(a) providing a culture of neural stem cells;

(b) incubating the culture of neural stem cells in the presence of an ovarian hormone;

(c) preparing cDNA from neural stem cells cultured without the ovarian hormone and neural stem cells of step (b), respectively; and

(d) comparing the cDNAs in step (c) to identify cDNAs that are induced or suppressed by the ovarian hormone.

The cDNAs identified by this method may code for factors which regulate neural stem cell numbers, and these factors can be used to increase neural stem cells in order to treat neurodegenerative diseases or conditions. Alternatively, they can be used as targets in drug discovery research for the identification of drugs which can lead to neural stem cell increase to treat these diseases or conditions.

Preferably, the induction or suppression level of the cDNA by the ovarian hormone is at least about two fold. The cDNA is more preferably induced or suppressed by the ovarian hormone by at least about four fold, still more preferably by at least about six fold, even more preferably by at least about eight fold, and most preferably by at least about ten fold.

In order to identify regulatory factors which play a primary role in neural stem cell increase, the neural stem cells are preferably incubated with ovarian hormone for less than 24 hours, more preferably for less than 12 hours, and most preferably for about 6 hours. It is contemplated that the ovarian hormone incubation may be shorter than 6 hours, for example 1, 2, or 4 hours, in order to identify the “immediately early” factors which are induced or suppressed quickly in response to ovarian hormone.

Another aspect of the present invention provides a method of treating or ameliorating a neurodegenerative disease or condition in a mammal, comprising administering an effective amount of an ovarian hormone to the mammal. Alternatively, an agent capable of increasing the level of an ovarian hormone, or a combination of such an agent and an ovarian hormone, can be employed to increase neural stem cells, thereby treating or ameliorating neurodegenerative diseases or conditions.

The neurodegenerative disease or condition may be a neurodegenerative disease, brain injury, or CNS dysfunction. The ovarian hormone or agent may preferably be administered to the brain, particularly a ventricle of the brain. Another preferred route of administration is administering the ovarian hormone systemically, particularly subcutaneously, topically or transdermally. Depending on the nature and severity of the disease or condition, it may be desirable to repeat the treatment more than once.

### DETAILED DESCRIPTION OF THE INVENTION

In the present invention, we discovered that ovarian hormones can increase the number of neural stem cells. This larger pool of neural stem cells can subsequently be used to generate more neural cells than would a population of stem cells without ovarian hormones. The neural cells, in turn, can be used in transplantations to compensate for lost or degenerate neural cells associated with neurodegenerative diseases or conditions. Alternatively, ovarian hormones can be added in vivo to increase neural stem cells, thereby increasing the production of new neurons or glial cells. Therefore, the present invention provides a method of increasing the number of neural stem cells, which can be used to treat or ameliorate neurodegenerative diseases or conditions.

The present invention also provides a method of identifying genes and others factors which regulate the number of neural stem cells. Once identified, the genes and factors can be used to increase the number of neural stem cells, and neural cells (neurons and glial cells) therefrom, in situ. The genes and factors can also be used as targets in the development of pharmaceutical agents which are capable of increasing neural stem cells by interacting with these targets in vivo.

Prior to describing the invention in further detail, the terms used in this application are defined as follows unless otherwise indicated.

Definitions

A “neural stem cell” is a stem cell in the neural cell lineage. A stem cell is a cell which is capable of reproducing itself. In other words, when a stem cell divides, at least some of the resulting daughter cells are also stem cells. The neural stem cells of the present invention, and their progeny, are capable of differentiating into all the cell types in the neural cell lineage, including neurons, astrocytes and oligodendrocytes (astrocytes and oligodendrocytes are collectively called glia or glial cells). Therefore, the neural stem cells are multipotent neural stem cells.

Preferably, the adult neural stem cells of the present invention refer to the neural stem cells located in or derived from the subventricular zone (SVZ) of the forebrain of adult mammals, which are different from the proliferating cells in the adult hippocampus. The SVZ and the subgranular layer (SGL) of the dentate gyrus of the hippocampus are two areas where neurogenesis has been described in adult mammalian brains. The SVZ is a thin layer of dividing cells persisting along the lateral wall of the lateral ventricles. New cells generated in the SVZ migrate as a network of tangentially orientated chains that converge on the rostral migratory stream (RMS) to reach the olfactory bulb, where they differentiate into local interneurons. In the dentate gyrus, new neurons are born in the SGL and migrate a short distance to differentiate into granule cells, which project axons to the CA3 region of the hippocampus.

The proliferating cells in the dentate gyrus are different from the adult neural stem cells in the SVZ for several reasons. First, the cells from the dentate gyrus do not expand in response to FGF-2 and heparin sulfate. Thus, when brain tissue is removed from the dentate gyrus and cultured in vitro, neurospheres can only be generated when EGF is added to the culture of dentate gyrus cells, while the combination of FGF-2 and heparin sulfate is not effective. In contrast, cells from the SVZ form neurospheres in either EGF or FGF-2/heparin sulfate containing medium.

Second, only a small percentage (no more than 10%) of the dentate gyrus-derived neurospheres are multipotent and capable of giving rise to all three kinds of neural cells, neurons, astrocytes and oligodendrocytes. The majority of these neurospheres (at least 90%) can only form astrocytes and oligodendrocytes. 99% of the SVZ-derived neurospheres, however, give rise to all three kinds of neural cells.

Third, it is well documented that proliferating cells in the SVZ and dentate gyrus respond differently to external stimuli. For example, corticosterone dramatically decreases cell proliferation in the dentate gyrus while having no effect on the SVZ proliferating cells (Alonso, 2000). Estrogen has also been reported to stimulate proliferation in the dentate gyrus but not the SVZ (Tanapat et al., 1999). Therefore, ample evidence indicates that the proliferating cells in the dentate gyrus are different from the multipotent neural stem cells in the SVZ.

“Pass 1 neural stem cells” are neural stem cells which have been passaged once in culture. Typically, neural stem cells can be obtained from an embryo or an adult brain tissue (for example the subventricular zone of the forebrain) and plated as a primary culture (see, for example, U.S. Pat. No. 5,750,376). The primary culture can then be dissociated and re-plated. The resulting cells, which have been passaged once in culture, are called the pass 1 neural stem cells.

A “neurosphere” is a group of cells derived from a single neural stem cell as the result of clonal expansion.

A “neural cell”, as used herein, refers to a neuron or glia.

An “ovarian hormone” is a hormone which is made by or present in the ovary. Preferably, the ovarian hormone is an estrogen or progestin, or the combination of both. In addition, the term “ovarian hormone” also refers to any other natural or synthetic substance which corresponds to a hormone made by or present in the ovary and is capable of inducing neural stem cell increase as determined by the methods described herein.

An “estrogen” is an “estrogenic” substance, i.e., a substance which is capable of inducing female characteristics in a mammal or activating the estrogen receptor. The estrogen is preferably a female steroid hormone with 18 carbons. The estrogen is more preferably estriol, estrone or estradiol, and most preferably β-estradiol. In addition, the term “estrogen” also refers to any other natural or synthetic estrogenic substance which is capable of stimulating neural stem cell proliferation as determined by the methods described herein. Examples of estrogens commonly used in the pharmaceutical industry include, but are not limited to, ethinyl estradiol, diethyl stilbestrol (DES), dimethyl stilbestrol (DMS), mestranol, Premarin® (conjugated estrogens), estropipate, tamoxifen, nafoxidin, raloxifene, droloxifene and phenol red.

A “progestin” is any substance which is capable of promoting the secretory changes in the uterine endometrium normally induced by progesterone, or activating the progesterone receptor. The progestin is preferably progesterone or 17-α-hydroxyprogesterone, and more preferably progesterone. However, the term “progestin” is contemplated to include any other natural or synthetic substance which has the progestin activity described above and which is capable of stimulating neural stem cell proliferation as determined by the methods described herein. In particular, progestins include the substances commonly used in the place of progesterone by the pharmaceutical industry, such as medroxyprogesterone acetate, norethindrone, norethindrone acetate, and norgestrel.

A “gene which participates in neural stem cell increase” is a gene the expression of which in neural stem cells is induced or suppressed during the process of ovarian hormone-induced neural stem cell increase.

A “neurodegenerative disease or condition” is a disease or medical condition associated with neuron loss or dysfunction. Examples of neurodegenerative diseases or conditions include neurodegenerative diseases, brain injuries or CNS dysfunctions. Neurodegenerative diseases include, for example, Alzheimer's Disease, Multiple Sclerosis (MS), macular degeneration, glaucoma, diabetic retinopathy, peripheral neuropathy, Huntington's Disease, Amyotrophic Lateral Sclerosis, and Parkinson's Disease. Brain injuries include, for example, stroke (e.g., hemorrhagic stroke, focal ischemic stroke or global ischemic stroke) and traumatic brain injuries (e.g. injuries caused by a brain surgery or physical accident). CNS dysfunctions include, for example, depression, epilepsy, neurosis and psychosis.

“Treating or ameliorating” means the reduction or complete removal of the symptoms of a disease or medical condition.

An “effective amount” is an amount of a therapeutic agent sufficient to achieve the intended purpose. For example, an effective amount of ovarian hormones to induce an increase of neural stem cells is an amount sufficient to in crease the number of the neural stem cells of interest, in vivo or in vitro. An effective amount of an ovarian hormone to treat or ameliorate a neurodegenerative disease or condition is an amount of the ovarian hormone sufficient to reduce or remove the symptoms of the neurodegenerative disease or condition. The effective amount of a given therapeutic agent will vary with factors such as the nature of the agent, the route of administration, the size and species of the animal to receive the therapeutic agent, and the purpose of the administration. The effective amount in each individual case may be determined empirically by a skilled artisan according to established methods in the art.

The effect of Ovarian Hormones on Neural Stem Cells

In the present invention, we discovered that ovarian hormones can lead to an increase of neural stem cells. It was first discovered that pregnant female mice and virgin female mice of the same age displayed different numbers of neural stem cells (Example 1), suggesting that female hormones associated with pregnancy may have an impact on the number of neural stem cells.

Since pregnancy is accompanied by many hormonal and non-hormonal changes in the physiology of the animal, we then determined if ovarian hormones in non-pregnant mice would influence the number of neural stem cells. As shown in Example 2, ovarectomy resulted in a significant decrease in the number of neural stem cells, indicating that ovarian hormones stimulated production, or reduced decrease, of neural stem cells.

Ovarian hormones are hormones made by or present in the ovary. The most abundant ovarian hormones are estrogens and progestins, the two hormones made by ovary. There are three main estrogens, β-estradiol, estrone and estriol. The principle estrogen secreted by the ovaries is β-estradiol, the estrogenic potency of which is 12 times that of estrone and 80 times that of estriol. Estrogenic potency is typically determined as the ability to induce the development of female characteristics in animals. Alternatively, estrogenic potency can also be determined as the ability to bind to and activate the estrogen receptor (for example see Baniahmad et al., 1995).

The naturally occurring progestins are progesterone and 17-α-hydroxyprogesterone. 17-α-hydroxyprogesterone has essentially the same biological activities as progesterone, but its abundancy is much lower. By far the most important function of progesterone is to promote secretory changes in the uterine endometrium during the latter half of the female cycle, thus preparing the uterus for implantation of the fertilized ovum. The activity of a progestin may also be determined as its ability to bind to and activate the progesterone receptor in an in vitro assay similar to that for estrogen (for example see Baniahmad et al., 1995).

This is the first time ovarian hormones are found to act on neural stem cells. It has been previously reported that estrogen had a cytoprotective effect on neural cells, and this effect can be distinguished from a mitogenic action (U.S. Pat. No. 5,843,934). Estrogen has also been reported to promote the recruitment and decrease the turnover of new neurons in the adult female canary brain (Hidalgo at el., 1995). However, these results indicate that estrogen can protect pre-existing terminally differentiated neural cells such as neurons, rather than exerting any biological functions on neural stem cells.

It was recently reported that estrogen can induce a transient increase in the number of new neurons in the dentate gyrus of adult female (Tanapat et al., 1999). However, this article also reported that there was no mitotic activity in the subventricular zone in response to estrogen. Since neural stem cells are primarily present in the subventricular zone of adult mammals, this article suggests that estrogen does not induce proliferation of neural stem cells. Tanapat et al. does not disclose any other effect of estrogen on the cells in the subventricular zone, and thus provides no information in regard to the number of neural stem cells in response to estrogen or any other ovarian hormone.

Accordingly, the present invention shows for the first time that ovarian hormones result in an increase of multipotent neural stem cells. The present invention thus provides a method of increasing the number of neural stem cells to facilitate subsequent transplantation treatments. Ovarian hormone can also be used to increase stem cells in situ by administering the ovarian hormone to an animal, preferably a mammal.

It is contemplated that any estrogen or progestin can be used in the present invention. A combination of estrogen and progestin, or a combination of different estrogens/progestins, is also contemplated. In addition to the main natural estrogens described above, estrogens useful in the present invention also include, but are not limited to, ethinyl estradiol, diethyl stilbestrol (DES), mestranol, Premarin® (conjugated estrogens), estropipate, dimethyl stilbestrol (DMS), tamoxifen, nafoxidin, raloxifene, droloxifene and phenol red. Similarly, progestins also include, without being limited to, medroxyprogesterone acetate, norethindrone, norethindrone acetate, and norgestrel. In addition, any agent that is capable of increasing the level of an ovarian hormone can also be used.

This invention also provides a method for the identification of genes which regulate neural stem cell numbers. These genes can be identified by subtraction hybridization and the subsequent cloning of genes which are induced or suppressed by ovarian hormones. Preferably, the induction or suppression level by ovarian hormones of the cDNA encoded by the gene is at least about two fold. The cDNA is more preferably induced or suppressed by the ovarian hormones by at least about four fold, still more preferably by at least about six fold, even more preferably by at least about eight fold, and most preferably by at least about ten fold.

Both positive and negative regulatory factors for neural stem cells may be identified by using the present method. Positive factors will include, for example, members of the signal transduction pathway which leads to production or survival of stem cells, transcription factors which facilitate production or survival, and factors which inhibit differentiation. These factors will be induced by ovarian hormones. Conversely, negative factors will be suppressed by ovarian hormones and will include, for example, factors which promote differentiation and factors which inhibit cell cycle progression.

In order to identify regulatory factors which play a primary role in neural stem cell increase, the neural stem cells are preferably incubated with ovarian hormones for less than 24 hours, more preferably for less than 12 hours, and most preferably for about 6 hours. It is contemplated that the ovarian hormone incubation may be shorter than 6 hours, for example 1, 2, or 4 hours, in order to identify the “immediately early” factors which are induced or suppressed quickly in response to the ovarian hormone.

The present invention further provides a method of treating or ameliorating a neurodegenerative disease or condition by using an ovarian hormone, an agent that can increase the level of an ovarian hormone, or a combination of both. The ovarian hormone or ovarian hormone-increasing agent can be administered by any applicable route that results in an increase in neural stem cells. A preferred route of administration is administering to the brain, preferably to a ventricle of the brain, and most preferably to a lateral ventricle of the brain. Another preferred route is systemic administration, including, for example, subcutaneous, intravascular, intravenous, intramuscular, intraperitoneal, topical, transdermal, intradermal, oral, rectal, vaginal, nasal, and pulmonary (e.g. by inhalation) administrations. Subcutaneous, topical and transdermal administrations are particularly preferred.

The following examples are offered to illustrate this invention and are not to be construed in any way as limiting the scope of the present invention.

### EXAMPLES

In the examples below, the following abbreviations have the following meanings. Abbreviations not defined have their generally accepted meanings.

| | |
|---|---|
| ° C. = | degree Celsius |
| hr = | hour |
| min = | minute |
| μM = | micromolar |
| mM = | millimolar |
| M = | molar |
| ml = | milliliter |
| μl = | microliter |
| mg = | milligram |
| μg = | microgram |
| FBS = | fetal bovine serum |
| DTT = | dithiothrietol |
| PBS = | phosphate buffered saline |
| DMEM = | Dulbecco's modified Eagle's medium |
| α-MEM = | α-modified Eagle's medium |
| EGF = | epidermal growth factor |
| FGF = | fibroblast growth factor |
| SVZ = | subventricular zone |
| SGL = | subgranular layer |

### Example 1

### Increased Neural Stem Cell Numbers in Pregnant Female Mice

The numbers of neural stem cells in the forebrain of adult CD 1 mice were determined in pregnant mice and virgin mice in order to investigate the effect of female hormones. The entire subventricular zones of the forebrain (both hemispheres) of adult female mice were dissected, enzymatically dissociated and plated in defined culture medium in the presence of epidermal growth factor as described in U.S. Pat. No. 5,750,376. Seven to ten days later, the numbers of neurospheres, each of which is clonally derived from a single stem cell, were counted.

Two pregnant (gestation day 14) female mice were compared to two aged-matched virgin mice:

Number of neurospheres

| | (mean ± standard error of the mean) |
|---|---|
| Virgin mice | 473 ± 45 |
| Pregnant mice | 651 ± 31 |

Thus, the pregnant female mice had approximately 40% more neural stem cells than the virgin mice, indicating that female hormones which are elevated during pregnancy may have a positive effect on the number of neural stem cells.

### Example 2

### Ovarectomy Decreases Forebrain Neural Stem Cell Numbers

In order to further examine the role of ovarian hormones on the number of neural stem cells, the numbers of neural stem cells of the forebrain of adult female CD1 mice were examined in both ovarectomized mice and sham-operated controls. Eight days after the ovarectomy or sham operation, the entire subventricular zone of the forebrain of each animal was used to prepare neural stem cells as described in Example 1.

The result from five ovarectomized mice is compared to that from five sham-operated controls:

Number of neurospheres

| | (mean ± standard error of the mean) |
|---|---|
| Ovarectomized mice | 403 ± 27 |
| Sham-operated mice | 630 ± 85 |

Thus, ovarectomy resulted in a 36% reduction in the number of neural stem cells, indicating that female hormones of the ovary, including estrogen, have a positive effect on neural stem cell numbers.

### Example 3

### Ovarian Hormone Increases Neural Stem Cell Numbers in Vitro

To determine if ovarian hormones increase the number of neural stem cells in vitro, the effect of an ovarian hormone on neural stem cell cultures will be examined. Primary neural stem cells (from embryonic day 14 or adult subventricular zone) will be cultured in EGF for seven days to make neurospheres. These primary neurospheres will then be dissociated and re-plated in EGF to make pass 1 neurospheres.

Pass 1 neurospheres, either embryonic or adult, will be dissociated and plated (50,000 cell/ml) in culture medium containing EGF alone or the combination of EGF and an ovarian hormone. The culture is allowed to progress for seven days. To determine the number of neural stem cells in each culture, single spheres will be dissociated and plated in single wells of a 96 well plate in culture medium containing EGF only. After 7 days, the number of spheres which come from one single sphere will be counted. The data will indicate that in response to the ovarian hormone, the number of neural stem cells increases significantly.

We claim:

1. A method of increasing neural stem cell number, comprising providing an effective amount of an ovarian hormone to at least one neural stem cell under conditions which result in an increase in the number of neural stem cells.

2. The method of claim 1 wherein the neural stem cell is located in the brain of an animal.

3. The method of claim 2 wherein the neural stem cell is located in a subventricular zone of the brain.

4. The method of claim 1 wherein the ovarian hormone is administered to a ventricle of the brain.

5. The method of claim 1 wherein the ovarian hormone is administered systemically.

6. The method of claim 1 wherein the animal is an adult animal.

7. The method of claim 1 wherein the neural stem cell is cultured in vitro.

8. The method of claim 1 wherein the ovarian hormone is an estrogen.

9. The method of claim 1 wherein the ovarian hormone is a progestin.

10. The method of claim 1 wherein the ovarian hormone is a combination of estrogen and progestin.

11. A method of identifying a gene which participates in ovarian hormone induced neural stem cell increase, comprising:
(a) providing a culture of neural stem cells;
(b) incubating the culture of neural stem cells in the presence of an ovarian hormone;
(c) preparing cDNA from neural stem cells cultured without the ovarian hormons and neural stem cells of step(b), respectively; and
(d) comparing the cDNAs in step (c) to identify cDNAs which are induced or suppressed by the ovarian hormone.

12. The method of claim 11 wherein the induction or suppression is at least two fold.

13. The method of claim 11 wherein the culture of neural stem cells is incubated in the presence of the ovarian hormone for less than 24 hours in step (b).

14. The method of claim 11 wherein the culture of neural stem cells is incubated in the presence of the ovarian hormone for less than 12 hours in step (b).

15. The method of claim 11 wherein the culture of neural stem cells is incubated in the presence of the ovarian hormone for about 6 hours in step (b).

16. The method of claim 11 wherein the ovarian hormone is an estrogen.

17. The method of claim 11 wherein the ovarian hormone is a progestin.

18. The method of claim 11 wherein the ovarian hormone is a combination of estrogen and progestin.

19. A method of treating or ameliorating a neurodegenerative disease or condition in a mammal, comprising administering an effective amount of an ovarian hormone to the mammal.

20. The method of claim 19 wherein the disease or condition is brain injury.

21. The method of claim 19 wherein the brain injury is a stroke.

22. The method of claim 19 wherein the disease or condition is selected from the group consisting of Alzheimer's Disease, Multiple Sclerosis (MS), Huntington's Disease, Amyotrophic Lateral Sclerosis, and Parkinson's Disease.
